(19) United States
(12) Patent Application Publication
(10) Pub. No.: US 2023/0270530 A1
(43) Pub. Date: Aug. 31, 2023

(54) REMOVABLE PARTIAL DENTURE

(71) Applicant: REGENTS OF THE UNIVERSITY OF MINNESOTA, Minneapolis, MN (US)

(72) Inventors: John MADDEN, Minneapolis, MN (US); Paul OLIN, Minneapolis, MN (US)

(73) Assignee: REGENTS OF THE UNIVERSITY OF MINNESOTA, Minneapolis, MN (US)

(21) Appl. No.: 18/012,153

(22) PCT Filed: Jun. 23, 2021
(86) PCT No.: PCT/US21/38679

§ 371 Date: Dec. 21, 2022

Related U.S. Application Data
(60) Provisional application No. 63/043,294, filed on Jun. 24, 2020.

(51) Int. Cl. A61C 13/267; A61C 13/01; A61C 13/10; A61C 13/00

(52) U.S. Cl. CPC A61C 13/267; A61C 13/01; A61C 13/10; A61C 13/0004

(57) ABSTRACT

A removable partial denture comprises a frame sized and shaped to conform to a mouth inner surface, the frame including a projecting mounting structure; and a segment having a slot for slidably receiving the projecting mounting structure when the segment is assembled to the frame. A method of fabricating a removable partial denture comprises: acquiring 3D digital image data of a patient's mouth and dentition; creating a 3D digital model of a removable partial denture; fabricating a frame shaped to conform to an inner surface of the mouth from the 3D digital model, the frame including a mounting structure; fabricating a segment from the 3D digital model, the segment having a slot for slidably receiving the mounting structure; and inserting the mounting structure of the frame into the slot of the segment to assemble the segment to the frame. The segment can be a tooth segment or a base segment.

### CROSS-REFERENCES TO RELATED APPLICATIONS

This application claims priority to U.S. Patent Application No. 63/043,294 filed Jun. 24, 2020.

### STATEMENT REGARDING FEDERALLY SPONSORED RESEARCH

Not Applicable.

### FIELD OF THE INVENTION

The removable partial denture is a removable dental prosthetic that replaces some, but not all the teeth in the dental arch. For that reason, it may be more common than the complete denture. This invention introduces a simplified workflow that expedites the clinical and laboratory steps of the manufacturing process of the removable partial denture and ultimately renders a product that could be produced directly from intraoral scans and three dimensional (3D) printing alone eliminating the need for stone models and heat cured acrylic entirely.

### BACKGROUND

A partial denture is a prosthesis that replaces one or more, but not all of the natural teeth and supporting structures. It is supported by the teeth and/or the mucosa. It may be fixed (i.e., a bridge) or removable. A removable partial denture (RPD) is a partial denture that can be removed and replaced in the mouth by the patient. RPDs are generally indicated for partially edentulous patients who cannot have fixed prostheses due to, for example, health conditions, cost or aesthetics considerations, and the extent and position of the edentulous span. RPDs are supported and retained by the remaining natural teeth (referred to as “abutment teeth”), tissue and/or implants.

Historically, the partial denture has been prototyped and manufactured in a long and drawn out process requiring many long dental appointments and a large number of steps where error can be introduced in the clinic or laboratory. Two of the large sources of error are using stone (plaster) models and the warping of acrylic during heat processing.

The first dental appointment traditionally begins with a set of alginate impressions of the patients remaining teeth. These impressions are then made into stone (plaster) models. The models are then analyzed using a process called “surveying”. This is used to determine the ideal path of insertion for the proposed partial denture. At this time, the rest seats for the frame are also planned and incorporated into the design.

After the design is planned, the patient has their second appointment. At this appointment, the rest seats and guide planes are prepared in the patient's teeth using a dental drill. New impressions are taken using alginate, and stone (plaster) models are poured again.

Both the models with the proposed design and the final models from the second appointment are sent to the laboratory for a chrome cobalt alloy metal frame to be fabricated. To fabricate the metal frame, a wax pattern of the frame is made on the stone model using pre-made wax pieces specifically grid work, clasps, and major connectors. The wax patterns are formed to fit the dental arch and match the proposed design.

The frame is then removed from the cast and invested. It is typically cast out of cobalt chrome alloy. After it is cast, the frame is divested and laboriously finished, polished, and fit back to the stone model. It is then returned to the dentist to be fit in the patient's mouth.

After the 3rd dental appointment (when the frame is fit in the patient's mouth), it is returned to the dental lab for “wax-rims” to be attached to the edentulous spaces on the frame. The frame with the wax-rim is then returned to the dentist for the jaw relations appointment.

At the 4th appointment, the wax-rims are adjusted to determine the correct vertical dimension of occlusion (VDO) and record the correct jaw relations or bite. All the components are then sent back to the laboratory so the prototype tooth arrangement can be made with pre-made carded denture teeth (relatively expensive).

At the 5th dental appointment, the dentist and the patient approve the proposed tooth arrangement and make any adjustments. If necessary, a new bite is taken and the case is remounted and adjustments are made in the laboratory.

After the prototype tooth arrangement is finalized, the model, the frame, and the tooth arrangement are flasked in stone (plaster) making a two-part mold. This mold is used to pack polymethyl methacrylate (PMMA acrylic) into the correct shape around the frame and secure the teeth.

After curing for 8 hours at 165° F., the acrylic is solid and is divested from the mold. It is then trimmed, finished, and polished to be delivered to the patient.

At the 6th appointment, the final removable partial denture is fitted to the patients mouth making any last minute adjustments. After wearing this for several days, there may be 1-2 follow-up appointments for adjusting sore spots.

It is estimated that some 12 million people in the United States are fully edentulous. On their path to losing all their teeth, they most likely have worn one or several removable partial dentures, leading to millions of these dental appliances being made in the United States each year.

The fabrication of removable partial dentures as described above often involves a complicated traditional workflow accompanied by ample room for human error. Furthermore, design-induced errors present themselves in abundance as the rudiments of traditional design become less and less familiar to the general dentist and technician. This is largely due to the fact that the basics of the partial denture are complex and the hours and repetition required to master these concepts are not found in today's dental education.

In addition to faulty design cues, many errors come from the traditional way RPD production uses heat-activated polymethyl methacrylate denture-base resin pressed to stone. These stone models are duplicated several times throughout the course of the fabrication introducing error and discrepancy with each duplication. The crippling tendency of acrylic shrinkage and contraction during thermal polymerization and the error of expansion and contraction of stone amplified over several layers of duplication yields frustrating results. However frustrating, the field of removable prosthodontics has been employing this wax-loss pattern and flasking technique for over a century. A step forward to replacing stone casts and pressed packed acrylics with digital design and 3D printing is long overdue.

In addition to detrimental effects of duplicating stone models and acrylic shrinkage, many tedious patient-dentist visits are inevitable to acquire physical records of oral anatomy to ensure proper fitment on the day of delivery. There are usually 3-5 appointments with the introduction of large error or failure at each. Each appointment also is separated by many calendar days if not weeks and along with time for shipping.

What is needed therefore is an improved method for fabricating a removable partial denture.

### SUMMARY OF THE INVENTION

In one aspect, the present disclosure provides a removable partial denture comprising: a frame sized and shaped to conform to a mouth inner surface, the frame including a projecting mounting structure; and a segment having a slot for slidably receiving the projecting mounting structure when the segment is assembled to the frame. The segment may be a tooth segment or a base segment. The segment(s) ‘draw’ or have slidability onto the mounting structure. The slidability or draw may have a path of insertion selected from buccal, anterior, posterior, or occlusal directions.

In one embodiment, the projecting mounting structure comprises an I-beam. A longitudinal axis of the I-beam can extend distally to mesially. A longitudinal axis of the I-beam can extend buccally to lingually. The I-beam can comprise opposed end plates connected by a midsection, the midsection can include a channel, the segment can include a throughhole, and the channel and the throughhole can be aligned when the segment is assembled to the frame.

In one embodiment, a fastener is positioned in the channel and the throughhole for immobilizing the segment on the frame when the segment is assembled to the frame. The fastener can comprise a cured resin. The fastener can comprise a pin. The channel and the throughhole can be aligned to form a passageway having a tapered inside diameter from one end to an opposite end of the passageway.

In one embodiment, a surface of the midsection of the I-beam can be textured adjacent the channel. The midsection can include at least one additional channel, the segment can include at least one additional throughhole, and each additional channel can be aligned with one of the additional throughholes when the segment is assembled to the frame.

In one embodiment, the frame includes at least one additional projecting mounting structure, and the removable partial denture includes at least one additional segment, each additional segment having a slot for slidably receiving one of the additional projecting mounting structures when the additional segments are assembled to the frame. In one embodiment, the frame is an implant bar.

In one embodiment, the removable partial denture further comprises at least one additional segment coupled to the frame. In one embodiment, the removable partial denture further comprises a clasp retainer attached to the frame. The clasp retainer can comprise an I bar clasp. The clasp retainer can comprise a circumferential clasp. The clasp retainer can comprise a wrought wire clasp. The wrought wire clasp can be embedded in a base section of the frame, wherein the base section is below a tooth segment. The wrought wire clasp can include a section that matingly engages corresponding structure in the base section of the frame.

In one embodiment, the frame is formed by 3D printing, and the segment is formed by 3D printing or milling.

In another aspect, the present disclosure provides a method of fabricating a removable partial denture. In one version, the method comprises: (a) acquiring 3D digital image data of a patient's mouth and dentition; (b) creating a 3D digital model of a removable partial denture to be fabricated; (c) fabricating a frame sized and shaped to conform to an inner surface of the mouth from the 3D digital model, the frame including a projecting mounting structure; (d) fabricating a segment from the 3D digital model, the segment having a slot for slidably receiving the projecting mounting structure; and (e) inserting the projecting mounting structure of the frame into the slot of the segment to assemble the segment to the frame. The segment may be a tooth segment or a base segment. Step (d) can comprise fabricating additional segments from the 3D digital model. Step (c) can comprise fabricating the frame using 3D printing. Step (d) can comprise fabricating the segment using 3D printing or milling.

In the method, step (c) can comprise fabricating the frame such that the projecting mounting structure comprises an I-beam. Step (c) can comprise fabricating the frame such that the projecting mounting structure comprises an I-beam having opposed end plates connected by a midsection, wherein the midsection includes a channel, and step (d) can comprise fabricating the segment such that the segment includes a throughhole, and step (e) can comprise inserting the projecting mounting structure of the frame into the slot of the segment such that the channel and the throughhole are aligned, and the method can further comprise: (f) positioning a fastener in the channel and the throughhole for immobilizing the segment on the frame. Step (f) can comprise curing a resin in the channel and the throughhole to form the fastener. In the method, step (c) can comprise fabricating the frame such that the midsection includes at least one additional channel, step (d) can comprise fabricating the segment such that the segment includes at least one additional throughhole, and each additional channel is aligned with one of the additional throughholes when the segment is assembled to the frame.

In one embodiment of the method, step (d) comprises fabricating at least one additional segment using 3D printing or milling, and coupling each additional segment to the frame. Step (c) can comprise fabricating the frame using 3D printing such that a clasp retainer is attached to the frame. The clasp retainer can comprise an I bar clasp. The clasp retainer can comprise a circumferential clasp. The method can further comprise embedding a wrought wire clasp in a base section of the frame, the base section being below a tooth segment. The wrought wire clasp can include a section that matingly engages corresponding structure in the base section of the frame.

In another aspect, the present disclosure provides a removable partial denture comprising: a frame sized and shaped to conform to a mouth inner surface; a tooth segment assembled to the frame; and a wrought wire clasp including a section that matingly engages corresponding structure in the frame or the tooth segment. In one embodiment, the section of the wrought wire clasp matingly engages corresponding structure in the frame. In another embodiment, the section of the wrought wire clasp matingly engages corresponding structure in the tooth segment. In another embodiment, the wrought wire clasp is embedded in a base section of the frame, the base section of the frame being below the tooth segment. The section of the wrought wire clasp can matingly engage corresponding structure in the frame. The section of the wrought wire clasp can matingly engage corresponding structure in the tooth segment. The wrought wire clasp can be embedded in a base section of the frame, wherein the base section of the frame is below the tooth segment. Thus, the wire clasp can matingly engage the base (which can be pink colored), the frame, or the tooth segment. The corresponding structure in the frame can comprise a cut out. The wire can include a relational element at a location where the wire leaves the base section. The relational element can comprise an eyelet.

In another aspect, the present disclosure provides a removable partial denture comprising: a base sized and shaped to conform to an inner surface of a patient's mouth; a tooth segment attached to the base; and a removable gingival shroud including a section that matingly engages corresponding structure in the base when the gingival shroud is assembled to the base or a frame of the removable partial denture. The section of the gingival shroud can include one or more protrusions, and the corresponding structure of the base can define one or more holes for receiving the one or more protrusions. The corresponding structure of the base can include one or more protrusions, and the section of the gingival shroud can define one or more holes for receiving the one or more protrusions. The gingival shroud can be dimensioned to hide the base from one viewing inside the patient's mouth when the removable partial denture is positioned in the patient's mouth and when the gingival shroud is assembled to the base or a frame of the removable partial denture. The base and the tooth segment can be substantially the same color. The base and the tooth segment can be fabricated as a single piece using 3D printing and/or milling.

In another aspect, the present disclosure provides a method of fabricating a removable partial denture. The method can comprise: (a) acquiring 3D digital image data of a patient's mouth and dentition; (b) creating a 3D digital model of a removable partial denture to be fabricated; (c) fabricating a base sized and shaped to conform to an inner surface of the mouth from the 3D digital model; (d) fabricating a tooth segment from the 3D digital model; and (e) fabricating a removable gingival shroud from the 3D digital model, the gingival shroud including a section dimensioned to matingly engage corresponding structure in the base when the gingival shroud is assembled to the base or a frame of the removable partial denture. The section of the gingival shroud can include one or more protrusions, and the corresponding structure of the base can define one or more holes for receiving the one or more protrusions. The corresponding structure of the base can include one or more protrusions, and the section of the gingival shroud can define one or more holes for receiving the one or more protrusions. The gingival shroud can be dimensioned to hide the base from one viewing inside the patient's mouth when the removable partial denture is positioned in the patient's mouth and when the gingival shroud is assembled to the base. The base and the tooth segment can be fabricated using 3D printing or milling as a single piece.

In another aspect, the present disclosure provides a removable partial denture comprising: a base sized and shaped to conform to an inner surface of a patient's mouth; a tooth segment attached to the base; and a clasp retainer, wherein the tooth segment and the clasp retainer are formed as one piece. The tooth segment and the clasp retainer can be fabricated as a single piece using 3D printing and/or milling. The base can be fabricated as a single piece using 3D printing or milling. The base can include an opening dimensioned to receive the tooth segment and the clasp retainer when the tooth segment is assembled to the base. The clasp retainer and the tooth segment can matingly engage in the opening in the base when the tooth segment is assembled to the base. The clasp retainer and the tooth segment can be positioned on top of the base when the tooth segment is assembled to the base. The clasp retainer can pass through a hole in the base.

In another aspect, the present disclosure provides a method of fabricating a removable partial denture. The method can comprise: (a) acquiring 3D digital image data of a patient's mouth and dentition; (b) creating a 3D digital model of a removable partial denture to be fabricated; (c) fabricating a base sized and shaped to conform to an inner surface of the mouth from the 3D digital model; and (d) fabricating a tooth segment and a clasp retainer as a single piece from the 3D digital model; and (e) assembling the tooth segment to the base. The base can be fabricated as a single piece using 3D printing. The base can include an opening dimensioned to receive the tooth segment and the clasp retainer when the tooth segment is assembled to the base.

The prior techniques for the design and geometry of an RPD framework design (such as the prior art process shown in FIG. 1) are a vestige of antiquated manufacturing techniques described in detail above. In the methods for fabricating a removable partial denture according of the present disclosure, heating premade wax patterns and shaping them to a stone model and casting is no longer necessary and neither is the engineering that originated with it.

It can be left behind because RPD frameworks can now be designed from scans in CAD software and then 3D printed directly out of a cobalt chrome alloy. The intermediary step of creating a wax pattern, investing, and casting can be eliminated entirely.

If the wax pattern is no longer necessary, the engineering of the framework inherent to the wax-loss induction casting method may be altered to fit the new manufacturing methods (3D printing and milling) and collection of data (scanning patient records and models). However, in making any changes to a one-hundred-year-old method that works well, we must be certain that any changes provide a distinct advantage.

In that regard (to that end), the changes proposed in the present disclosure provide the advantage of not requiring a stone dental model. This eliminates the physical shipping of records costing time, money, and mostly convenience to the patient and clinician. It also eliminates the error in expansion of the stone and distortion of vinyl polysiloxane (VPS) impressions.

In order to eliminate these errors and inconveniences and complete the RPD in fewer appointments, all the latest technology must be used in all stages of collection and manufacturing. First, the raw data must be collected using an intraoral scanner rather than with traditional VPS or alginate impression techniques.

After the scans are collected by the dentist, they can be sent to the laboratory for the design of the frame and the tooth segments. These pieces can be made using 3D printing. The two pieces can then be joined without the model and be returned to the dentist and the patient for delivery.

Using this new method and design of the present disclosure to create a modern dental device eliminates 1-3 appointments for the dentist and patient and leads to ultimate efficiency and cost savings in the laboratory.

The foregoing and other aspects and advantages of the invention will appear from the following description. In the description, reference is made to the accompanying drawings which form a part hereof, and in which there is shown by way of illustration example embodiments of the invention. Such embodiments do not necessarily represent the full scope of the invention, however, and reference is made therefore to the claims, drawings, and description herein for interpreting the scope of the invention.

### BRIEF DESCRIPTION OF DRAWINGS

FIG. 1 is a flowchart reviewing a traditional prior art removable partial denture process.

FIG. 2 is a flowchart showing a process of the present disclosure for fabricating a removable partial denture.

FIG. 3 shows a removable partial denture according to one non-limiting example embodiment of the present disclosure.

FIG. 4 shows a comparison of a step in the fabrication of a prior art removable partial denture (left) and a removable partial denture of the present disclosure (right).

FIG. 4A shows one embodiment of a flattened ridge gridwork suitable for use in a removable partial denture of the present disclosure, wherein the left view is a top, rear perspective view, and the right view is a side perspective view.

FIG. 4B shows another embodiment of a flattened ridge gridwork suitable for use in a removable partial denture of the present disclosure, wherein the left view is a top, rear perspective view, and the right view is a side perspective view.

FIG. 4C shows another embodiment of a flattened ridge gridwork suitable for use in a removable partial denture of the present disclosure, wherein the left view is a top, rear perspective view, and the right view is a side perspective view.

FIG. 5 shows a side perspective view of a frame of a removable partial denture according to another non-limiting example embodiment of the present disclosure.

FIG. 6 shows a side view of a frame of a removable partial denture according to another non-limiting example embodiment of the present disclosure.

FIG. 7 is a detailed cross-sectional view of a tooth segment mounting structure of the frame of the removable partial denture of FIG. 6.

FIG. 8 is a detailed cross-sectional buccal view of a group of tooth segment mounting structures of the removable partial denture of FIG. 6.

FIG. 9 is a side cross-sectional view of a tooth segment mounting structure of the frame of the removable partial denture of FIG. 6.

FIG. 10 is a partial side view of a removable partial denture according to another non-limiting example embodiment of the present disclosure.

FIG. 11 is a side view of a frame of a removable partial denture according to another non-limiting example embodiment of the present disclosure.

FIG. 12 is a perspective view of a frame of a removable partial denture according to another non-limiting example embodiment of the present disclosure.

FIG. 13 is a side cross-sectional view of tooth segments and a tooth segment mounting structure of a frame of a removable partial denture according to another non-limiting example embodiment of the present disclosure.

FIG. 14 is a perspective view of an I bar clasp suitable for use in a removable partial denture according to another non-limiting example embodiment of the present disclosure.

FIG. 15 is a perspective view of a circumferential clasp suitable for use in a removable partial denture according to another non-limiting example embodiment of the present disclosure.

FIG. 16A is a perspective view of a wrought wire clasp suitable for use in a removable partial denture according to another non-limiting example embodiment of the present disclosure.

FIG. 16B is a perspective view of another wrought wire clasp suitable for use in a removable partial denture according to another non-limiting example embodiment of the present disclosure.

FIG. 17 is a perspective view of another wrought wire clasp suitable for use in a removable partial denture according to another non-limiting example embodiment of the present disclosure.

FIG. 18 shows side views of other non-limiting wrought wire clasps suitable for use in a removable partial denture according to other non-limiting example embodiments of the present disclosure.

FIG. 19 shows a front, right perspective view of an implant bar without tissue or teeth.

FIG. 20 shows a completed implant bar of FIG. 19 from bottom view (left) and occlusal view (right).

FIG. 21 shows geometry or texture added to the implant bar of FIG. 19, but not aligned for insertion.

FIG. 22 shows three separate paths of insertion for the tooth segment on tooth segment mounting structure(s) of the implant bar of FIG. 19.

FIG. 23 shows a single path of insertion for the tooth segment on a tooth segment mounting structure for a complete arch of the implant bar of FIG. 19.

FIG. 24 shows in top view, a 3D printed removable partial denture having two tooth segments and integral framework, and in front side view, a gingival shroud dimensioned to engage the removable partial denture in a complementary fit according to another non-limiting example embodiment of the present disclosure.

FIG. 24A shows in top view in a patient's mouth, the gingival shroud of FIG. 24 assembled to the 3D printed removable partial denture of FIG. 24.

FIG. 25 shows a partial side view of a 3D printed removable partial denture having an integral tooth segment and clasp retainer according to another non-limiting example embodiment of the present disclosure.

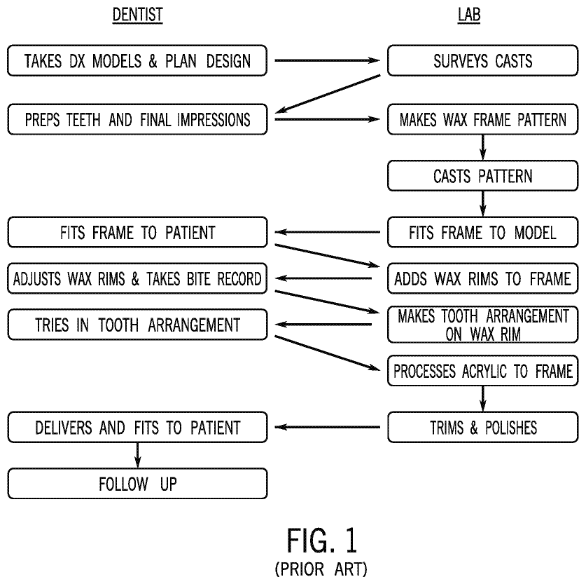

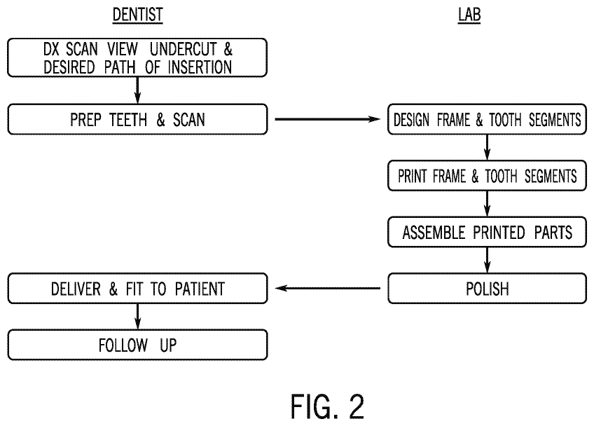

Like reference numerals will be used to refer to like parts from Figure to Figure in the following description of the drawings.

The invention will be better understood, and features, aspects and advantages other than those set forth above will become apparent when consideration is given to the following detailed description thereof. Such detailed description makes reference to the drawings.

### DETAILED DESCRIPTION OF THE INVENTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

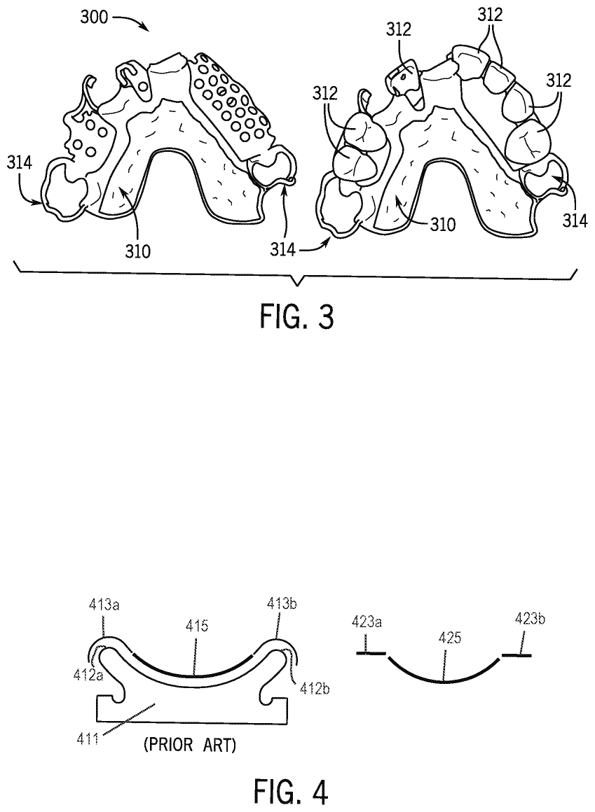

As stated above, to harness this technology, the shape, engineering, and geometry of the RPD framework must be altered from the traditional design. Furthermore, the material comprising the RPD of the present disclosure can be changed from cobalt chrome alloy to any number of materials including acetal, fiber reinforced composites)(Trilor®), polyether ether ketone (PEEK) (Pekkton®), polyaryletherketone (PAEK), nylon, or titanium. Each material changes the design and engineered strength slightly. However, the following basic design of a new two-part modern RPD of the present disclosure remains the same. The new design has two main components with some potential variations. Looking at FIG. 3, two newly engineered components that comprise a removable partial denture 300 according to one embodiment of the present disclosure are the frame 310 and the tooth segments 312. The frame 310 also includes circumferential clasp retainers 314 in this example embodiment.

### The Frame

Compared to prior frames, the change in the design of the frame may be divided into two sections: (1) changes to the grid under the edentulous spaces, and (2) changes to the assembly and connection of the clasps to the frame.

### Changes to the Grid Work in the Edentulous Areas

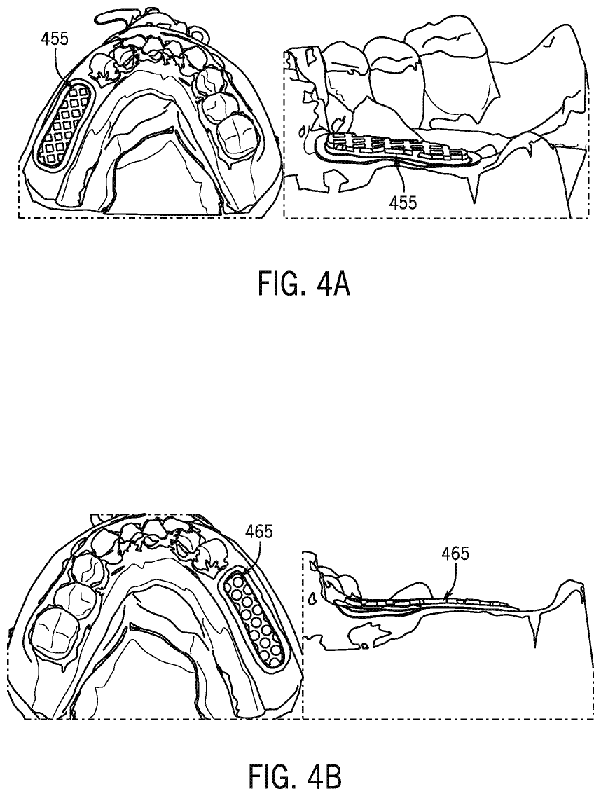

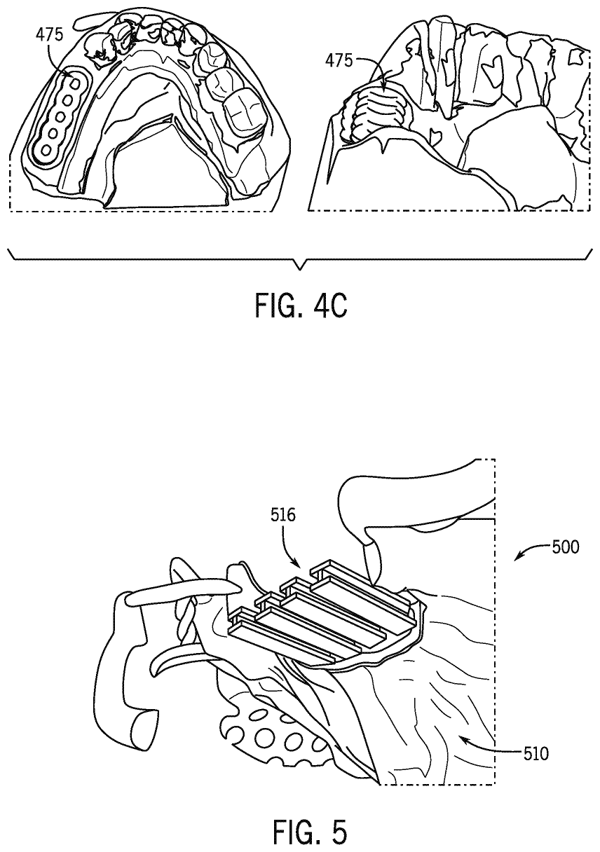

FIG. 4 shows a comparison of a step in the fabrication of a prior art removable partial denture (left) and a removable partial denture of the present disclosure (right). One change in the RPD device of the present disclosure compared to prior devices is in the frame's gridwork. The elimination of the prior art stone model 411 having curved gridwork 413a, 413b and connector 415 allows for the gridwork (which was previously curved over the ridges 412a, 412b of the stone model 411) to be straightened as in the straight bars 423a, 423b and connector 425 shown in a removable partial denture of the present disclosure (right) in FIG. 4.

There are other non-limiting embodiments for the gridwork for a removable partial denture of the present disclosure, such as plain (a simple flat plane), grid (square, shown at 455 in FIG. 4A), hole (small, shown at 465 in FIG. 4B), and torus ladder (shown at 475 in FIG. 4C). Any of these can be altered to allow for “slidability” (i.e., allowing the pieces to slide together as detailed below) when adapted to a horizontal flat plane rather than curved over the natural ridge.

As shown in FIG. 5, the curved gridwork 413a, 413b of the prior art of FIG. 4 is replaced with straight I-beam tooth segment mounting structures 516 on the frame 510 on a removable partial denture 500 according to another non-limiting example embodiment of the present disclosure. The tooth segment mounting structures 516 on the frame 510 allow the tooth segments (such as tooth segments 312 in FIG. 3) to be later attached in exact correct relation without the stone model (digitally only). Furthermore, the shape of I-beam tooth segment mounting structures 516 must be as such that it allows the tooth segment to be attached to the frame by same method (and keyed in precise relation). In certain situations, the ‘slidability’ can work without the gridwork (see FIGS. 4-4C) or “I-beam” component (see FIG. 5) being completely flat. It is only required that the piece can slide on and off cleanly for assembly. The gridwork can be curved in a direction that still allows ‘draw’ (rather than parallel) with the path of insertion of the tissue segment (pink acrylic resin). This especially applies if the tissue segmented is inserted from the most posterior distal extension.

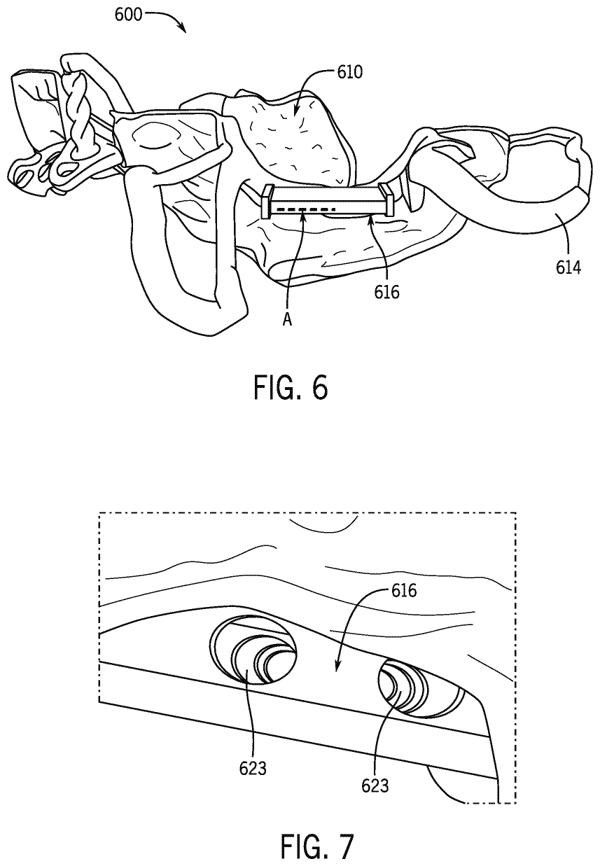

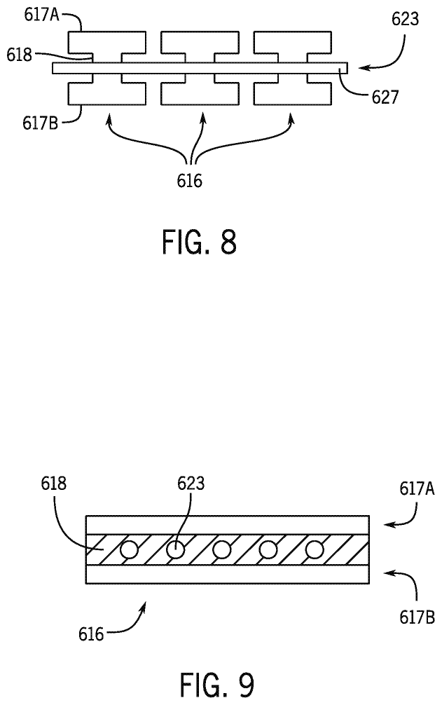

Turning now to FIGS. 6-9, there is shown a removable partial denture 600 according to another non-limiting example embodiment of the present disclosure. The removable partial denture 600 includes a frame 610 having an I-beam tooth segment mounting structure 616 on the frame 610 (which includes circumferential clasp retainer 614). In FIG. 6, a longitudinal axis A of the I-beam 616 extends buccally to lingually. However, a longitudinal axis of the I-beam may alternatively extend distally to mesially. In FIGS. 6 and 7, one I-beam tooth segment mounting structure 616 is shown on the frame 610. Alternatively, FIGS. 8 and 9 show that a plurality of I-beam tooth segment mounting structures 616 may be used on the frame 610. The I-beam tooth segment mounting structures 616 comprise opposed top and bottom end plates 617A and 617B connected by a midsection 618 that includes channels 623. The channels 623 align with a throughhole on the tooth segment when the tooth segment is assembled to the frame. A fastener positioned in the channels 623 and the throughhole immobilizes the tooth segment on the frame 610 when the tooth segment is assembled to the frame 610. In FIGS. 6-9, the fastener comprises a cured resin 624. The self-cure of light-cure injectable resin can be replaced with a solid pin fastener instead.

Thus, adherence of tooth segments to the frame 610 is provided by a series of coordinated channels 623 through the I-beam 616 that creates a tunnel where a self-curing or light-curing resin 624 or cement can flow. This channel and I-beam design is shown in FIGS. 6-9. Changing the frame 610 in this manner leads to a printed or milled tooth segment that can cleanly be inserted onto the frame 610 in correct relation using I-beam tooth segment mounting structures 616 on the frame 610. In traditional RPD design, a clasp or an I-bar clasp may come out of the acrylic segment, therefore adaptations can also be made in how the clasps attach to the frame.

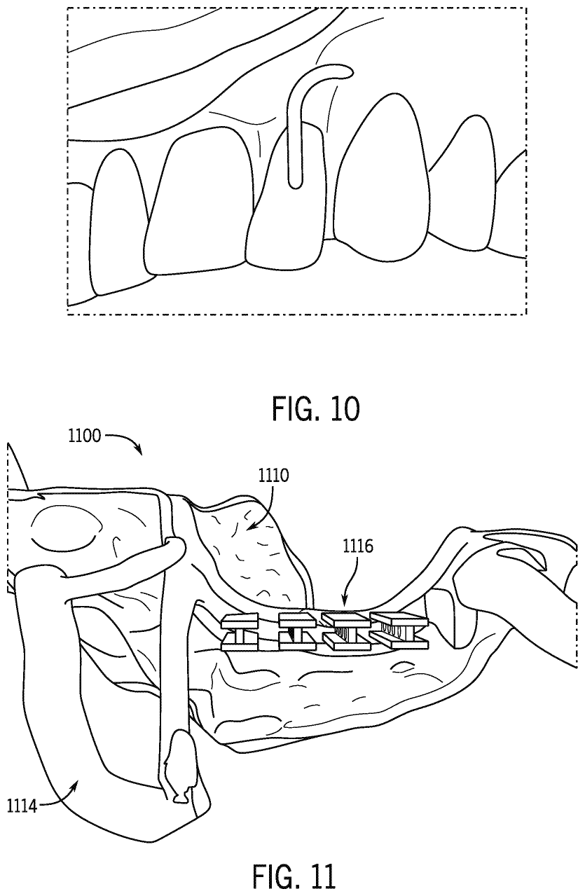

Using this new method, attachment of a regular “C” clasp or circumferential clasp 614 to the frame 610 will remain the same, but an I-bar clasp and a wrought wire clasp need to be attached to the frame 610 differently. To attach the I-bar clasp to the frame 610, it must be attached to a vertical plate near the guide plane of the edentulous area as shown in FIG. 10. Turning to FIG. 11, the removable partial denture 1100 includes a frame 1110 having I-beam tooth segment mounting structures 1116 on the frame 1110 which includes an I-bar clasp retainer 1114.

Assembling the clasps allowing the pieces to slide together is even more complex when incorporating a wrought wire clasp. The wrought wire clasp can be achieved in several ways. Attaching it to the frame 610 can be done by allowing a second channel the diameter of the wrought wire for it to pass through and be tack welded to. So in addition to a resin channel, there is a custom channel for the wrought wire. In addition to this method, the wrought wire can be attached by leaving relief in the tooth segment to cure the wire in place with self-cure or light cured resin.

### Implant Locator Attachments

Locator attachments are commonly found in partial denture applications. An implant with a locator attachments and housing can be processed into an acrylic of the removable partial denture 610. When done correctly, it may require a large amount of vertical dimension of occlusion (VDO), or freeway space. However, this is not often present in these cases.

The reason it requires so much space is because all the components are separate instead of combined as one piece. The metal housing, the nylon insert, and the 1+ millimeters of acrylic encapsulating the housing take up valuable space that would ideally be used for tooth arrangements.

With the printing of the components, as in one method of the invention, it is possible to print the housing as part of the frame and directly place the nylon insert in the frame. This creates over 2 millimeters of additional space circumferentially and aides in the ideal tooth arrangement.

### Retention of the Frame to the Tooth Segment (Edentulous Space)

Rather than having the I-beam 616 tapered buccal-lingually to assemble and adhere the tooth segments (with self-cure or light cure material 624 filling the channel 623), the I-beam 616 can have a buccal lingual taper or ramp on the vertical section of the I-beam 616.

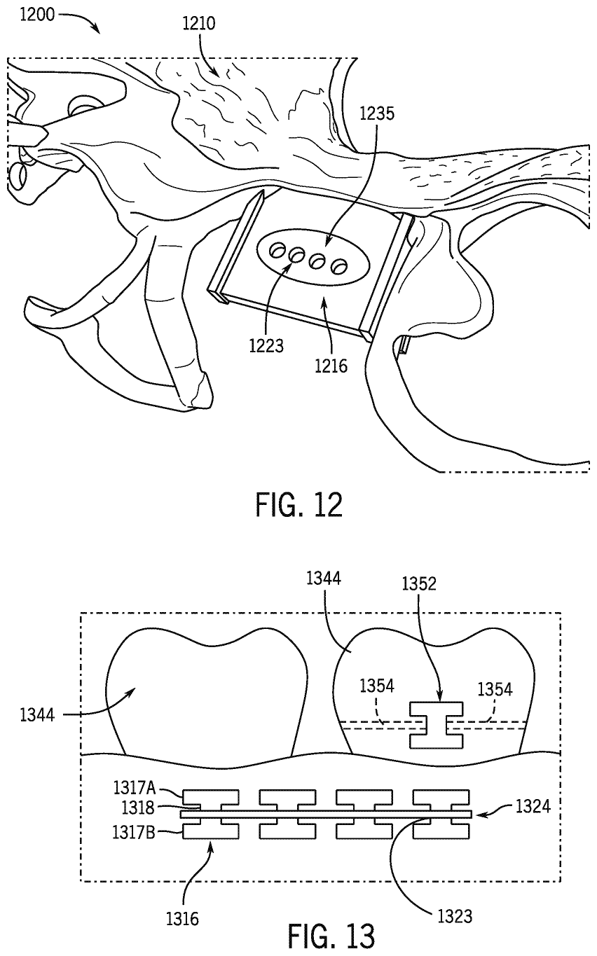

Another method of retaining the printed tooth segment is accomplished using the advantages that 3D printing metal naturally lend. Looking at FIG. 12, three-dimensional printing of metals allows elaborate textures 1235 to be embossed or debossed on the surface with ease and precision that is not possible by other means.

These textures when debossed on the vertical portion of the I-beam 1216 around the resin channels 1223 can provide the necessary micro-mechanical retention necessary to ‘adhere’ the tooth segment to the frame 1210 regardless of the frame material. This pattern could take many different forms, but a cross-hatched pattern or a ‘sandy’ surface debossed in a rectangular or ovoid area surrounding the resin channel is one non-limiting example design for the removable partial denture 1200.

Retention of the Metal Frame to the Tooth Segment with a Metal Saddle

Retention of the metal, acetal, or high-performance polymer frame to the tooth segment has its most advantageous manufacturing results when the tissue side of the edentulous area is not a part of the tooth segment but rather the RPD framework. In some cases, this metal tissue side or metal saddle was requested by the dentist most commonly when many metal ‘dummy teeth’ were incorporated into the frame and valuable space was limited. In this case, the overall design may save space, be stronger, and made more durable, but in the case of a metal frame, the tissue side of the appliance may be difficult to adjust.

Perhaps a preferred combination of these techniques would be with acetals and high-performance polymers. When applying this proposed hybrid method of assembly for either polymers or metal frames with a saddle, the milling times of metal frame or high-performance polymers can be reduced by up to 50%. On average, it takes a 3.5 to 4 hour mill time and reduces it to just under 2 hours. This is largely due to the mill removing much less material in the edentulous areas with less depth and detail. The advantage to this technique to small on-site manufacturing and high volume milling operations is enormous time savings and less in-sourcing and out-sourcing.

### CAD Generation

The method of combining a specialized framework with a tooth segment and corresponding resin channel through both requires a specific set of CAD operations. Firstly, the frame must be designed to meet the needs of the selected material. A proposed offset layer of 0.12-0.13 millimeters must be created around the I-beams (or chosen structure/member). Next, the tooth arrangement and gingiva must be designed (as one or two segments) and a Boolean subtraction must be done of the frames from the tooth segment. After this subtraction is completed, the resin channel pattern must be incorporated into both pieces and the resin channel pattern Boolean subtracted from both the tooth segment and the frame at the same time.

Now all three pieces are subtracted from each other and the two remaining pieces can be assembled and locked together.

### Additions to the Resin Channel and Connections for Assembly

The resin channel 623 can be tapered from one end to the next. The circumference of the channel should be larger at the opening than at the exit. This shape allows for pressure to build in the channel ensuring the channel is filled entirely and allowing for maximum strength.

Looking now at FIG. 13, it can be seen that a plurality of I-beam tooth segment mounting structures 1316 may be used on the frame. The I-beam tooth segment mounting structures 1316 comprise opposed top and bottom end plates 1317A and 1317B connected by a midsection 1318 that includes channels 1323. When the tooth segments 1344 are assembled to the I-beam tooth segment mounting structure 1316, each projecting mounting structure 1316 is slidably received within a slot 1352 of each tooth segment 1344 and the resin channels 1323 align with a throughhole 1354 in the tooth segment 1344. A fastener positioned in the channels 1323 and the throughhole 1354 immobilizes the tooth segment 1344 on the frame when the tooth segment 1344 is assembled to the frame. In FIG. 13, the fastener comprises a cured resin 1324. The self-cure or light-cure injectable resin can be replaced with a solid pin fastener instead. Thus, adherence of tooth segments 1344 to the frame is provided by a series of coordinated channels 1323 through the I-beam 1316 and a throughhole 1354 in the tooth segment 1344 that creates a tunnel where a self-curing or light-curing resin 1324 or cement can flow. Optionally, one tooth segment 1344 can be mounted to the mounting structure 1316 and additional tooth segments can be coupled to the tooth segment that is mounted to the mounting structure 1316.

Adhesion of the base section to the frame, or tooth segment to the base section, may be achieved using, without limitation: cementing, luting, adhesion by adding light cure or self-cure resins or composites, other adhesives or silanes, and may include micro retentive features or priming. The present disclosure includes both methods of the base section (tissue segment) and tooth segment being separate, or the base section and the tooth segment as one piece. The advantage of them being separate is that it allows for a resin channel opening in the tooth sockets where the base can be luted to the frame with resin, afterwards the teeth can be luted in place as well. Thus, the segment having a slot for slidably receiving the projecting mounting structure when the segment is assembled to the frame may be a base segment or a tooth segment.

### Clasp Retainers

Three example clasp retainers are: (1) “I bar” clasp; (2) circumferential (circle or Akers) clasp; and (3) wrought wire clasps. Clasps may engage an external surface of an abutment tooth in a natural undercut or in a prepared depression. There are two main classes of clasps: (i) those that approach the undercut from above the height of contour (suprabulge retainers), and (ii) those that approach the undercut from below (infrabulge retainers).

### I Bar

The I bar style clasp assembly can be cast out of cobalt chrome alloy and can be a part of the rest of the cobalt chrome alloy frame (cast or printed). They are typically one piece and the same material, usually cobalt chrome alloy.

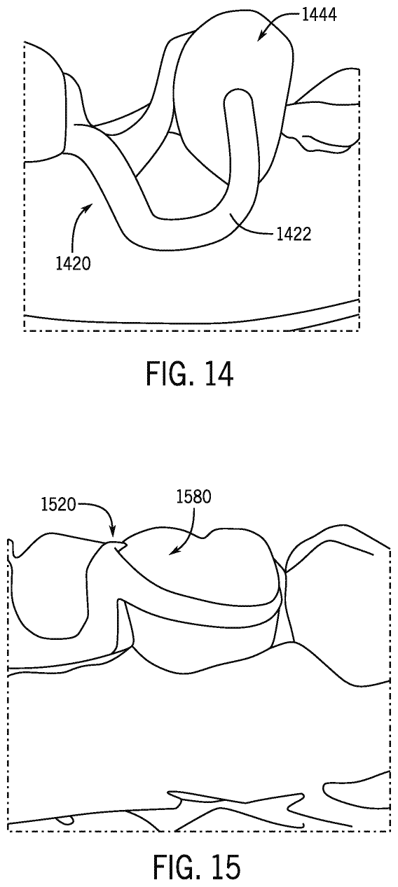

Looking at FIG. 14, it can be seen that the clasp arm 1422 is cast cobalt chrome alloy and swoops very low coming from underneath the tooth 1444 it engages. In order to be used in our proposed design, the clasp 1420 must connect to the frame in a different place than usual, a different place than shown here. The whole assembly must be moved forward and allow the base piece to draw off and on.

### Circumferential (Circle or Akers) Clasp

Looking at FIG. 15, the cast Akers clasp 1520 is also a single piece of cobalt chrome alloy that is cast with the rest of the framework as one piece. The difference is it starts higher on the frame near the height of contour of the abutment tooth 1580 and appears shorter. This style of clasp requires little to no adaptation to incorporate our proposed design. This clasp is typically a suprabulge retainer.

### Wrought Wire Clasp

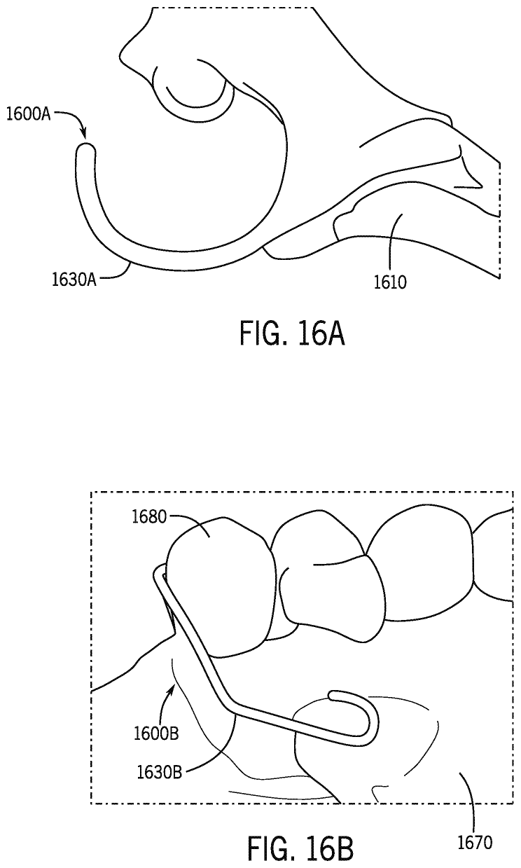

The wrought wire clasps 1600A or 1600B in FIGS. 16A and 16B require the most understanding and explanation with regard to our RPD of the present disclosure. The clasp 1600A or 1600B is made of a separate piece of wrought steel wire that is shaped by hand to the stone model. This clasp is typically a infrabulge retainer. After being shaped, the wire 1630A is soldered to the frame 1610 as in FIG. 16A or the wire 1630B is embedded in the acrylic 1670 as in FIG. 16B. It is very similar in size and shape to the cast Akers clasp previous described. A bent wire is soldered to a cobalt chrome alloy frame or embedded in acrylic. This applies to RPDs that include a metal frame and all acrylic RPDs. Typically, a stone model is required to shape the wire. One purpose of our RPD concept is to use no stone model. A machine was developed to bend such a wire using a CAD designed wire. Therefore, producing the wire in the correct shape for the abutment tooth 1680 without the stone model is possible. However, attaching it correctly in the correct orientation can be a problem. However, if we use our method described herein, this is solved.

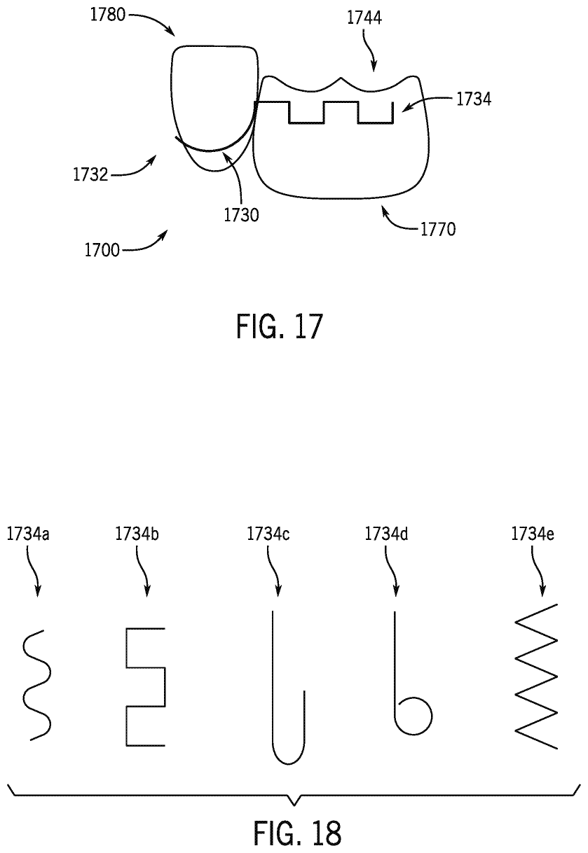

Turning to FIG. 17, in our method, the wrought wire clasp assembly problem is solved by embedding the wire 1730 into the base section 1770 of the frame underneath the tooth segment. The wire 1730 has an end 1732 engaging the abutment tooth 1780. The wire is 1730 bent with a tail 1734 that has a specific design running parallel (horizontally) to the bottom of the tooth segment 1744 that allows it only to be adhered and related in one direction. This eliminates the need for the stone model. This same method can be applied to an all acrylic RPD as well. Relating a wire into an acrylic base without a stone model is a key advantage of our RPD of the present disclosure. The wire is bent with a tail in a specific geometry that matches the cut out somewhere in the acrylic base that allows it only to go in one direction. Where the wire leaves the base it may also require another relational element or stop, like an eyelet. The wire may be embedded in the base (tissue), the tooth segment, the frame itself, or any combination thereof.

The wire can be bent so it may only be inserted and embedded into the base section 1770 in one way. The cut out in the base section is a Boolean subtraction of sorts of the wire itself. This may require an ‘eyelet’ where the wire exits the base to aide in orientation of the wire. This may include any shape of common ‘tail’ or ‘loop’ on the end of the wire that is embedded in the base. The shape of the tail may include common ‘s’ shaped tail 1734a, square tail 1734b, hairpin tail 1734c, loop tail 1734d, or triangular tail 1734e as shown in FIG. 18.

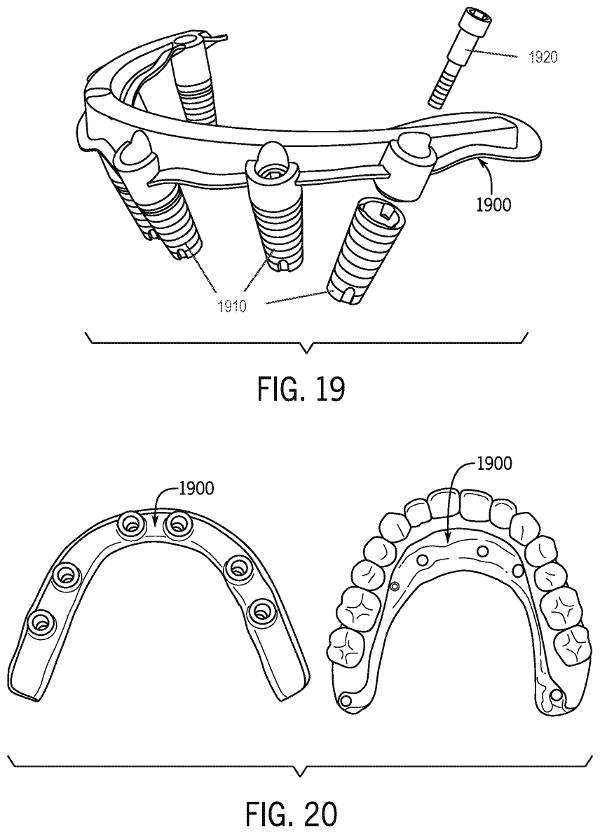

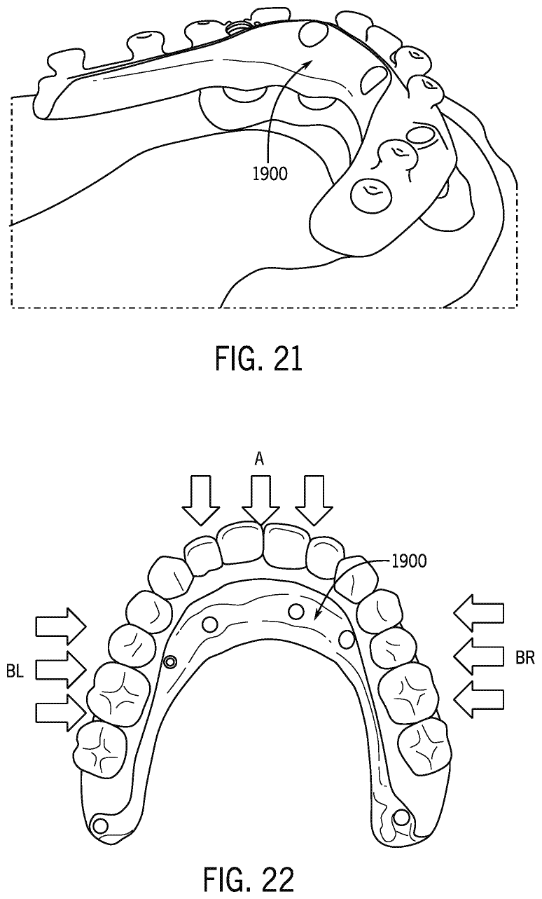

Referring now to FIGS. 19-23, the assembly method of the present disclosure also applies to “Montreal style” or other implant bars (typically used with implants 1910 and screws 1920) where one or multiple tissue and tooth segments (such as 1344 in FIG. 13) can slide onto an I-beam tooth segment mounting structure (such as I-beam 1316 in FIG. 13) of the implant bar 1900 in a similar fashion. This may include a different path of insertion for each tissue and tooth segment pair. In FIGS. 19-23, it can be seen that the implant bar 1900 in this configuration is similar to a partial framework with an all metal saddle. Therefore, the assembly method of the present disclosure can be applied to this prosthesis as well. The implant bar itself in its current configuration looks some like FIG. 19 and the completed prosthesis in FIG. 20. FIG. 21 shows how geometry can be added to the bar 1900 for mechanical retention. If that mechanical retention was aligned properly like in the I-beam tooth segment mounting structures of the present disclosure or something similar, slidability can be achieved to assemble this prosthetic as well from the buccal, anterior, or occlusal directions.

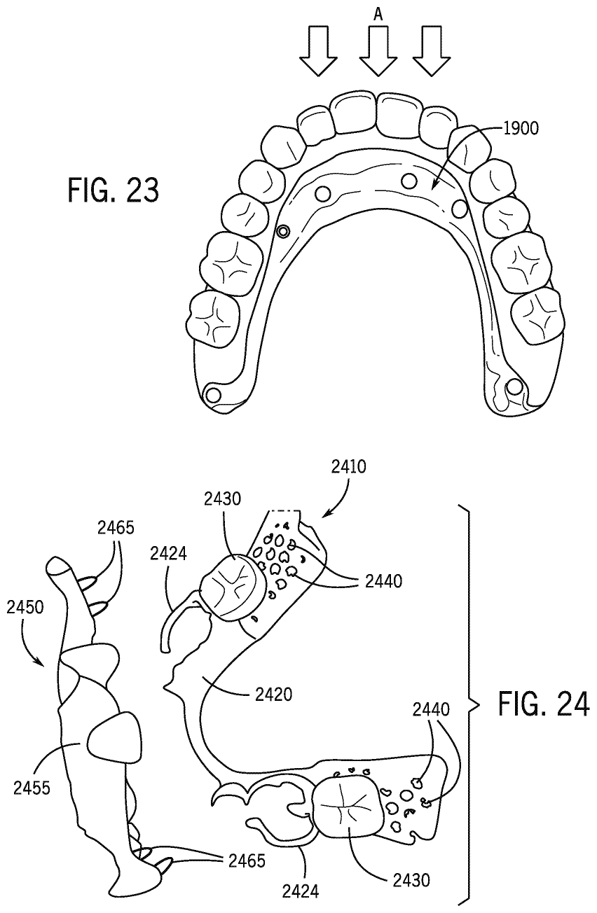

One possibility is to break the arch of the tissue segment into three separate pieces and have three separate sets of I-beam tooth segment mounting structures with three separate insertion directions: Buccal Left BL (4 posterior teeth), Anterior A (6 anterior teeth), and Buccal Right BR (four posterior teeth). The paths of insertion for the tooth segments would look as shown in FIG. 22. A single path of insertion is also possible for the entire arch as shown in FIG. 23, although it allows for decreased manufacturing tolerances. This can be achieved from both the labial (anterior) direction A or more easily from the occlusal direction (straight down).

### Gingival Shroud

One example embodiment of the digital partial denture will include a frame and a tooth segment that are the same color and material. This allows them to be one single piece. This method is currently commonly used with acetal and polymethylmethacrylate (PMMA) partial dentures when digitally designed. For example, acetal or PMMA may be colored A2 (a common tooth shade). The advantage to this color is that it allows the frame and the tooth segment to be one piece adding strength, convenience, and ease in manufacturing. While this may be strong and efficient, it lacks in aesthetics and does not meet the expectations of most dentists and patients.

An RPD most commonly comprises a pink base with metal clasps and white tooth segment. This is the expectation of most dentists and patients. If the RPD is all white or mostly white, the RPD does not meet the current set of standards and expectations. In accordance with one example embodiment of the invention, a pink gingival shroud component fixes this problem with prior RPDs. This gingival shroud provides a way to meet dentist's and patient's expectations with ease using available 3D printing resins with minimal software ‘work arounds’. The gingival shroud is a pink gingival component that slides over and around the teeth segments on the frame of an RPD. Among other things, this gingival shroud component is made for cosmetic reasons.

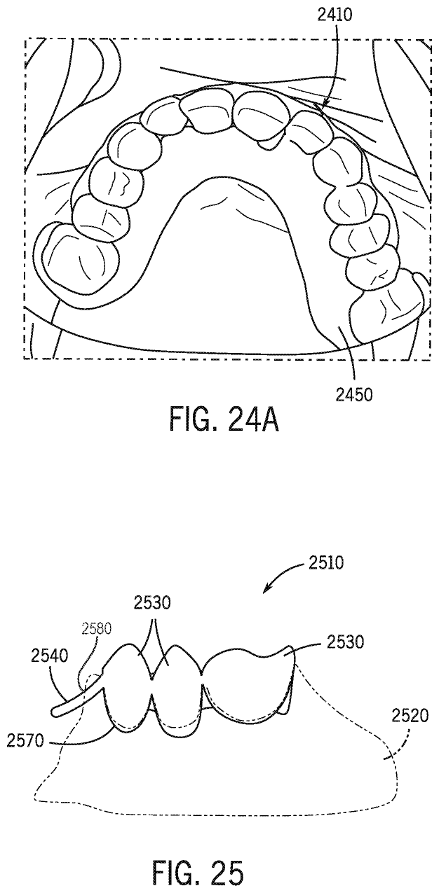

Looking at FIG. 24, there is shown a removable partial denture 2410 having base framework 2420 with clasp retainers 2424 and tooth segments 2430. In the removable partial denture 2410, the white tooth segments 2430 can be fabricated as one piece with the RPD framework 2420 (whether metal, acrylic, porcelain or other material). In accordance with the invention, a gingival shroud 2450 having a generally arch-shaped body 2455 is included as a separate piece simulating the pink gingival tissues. The gingival shroud 2450 may be merely cosmetic or essential for proper support and adaptation of the RPD to the supporting and surrounding oral tissues. The gingival shroud 2450 is dimensioned to hide the base from one viewing inside the patient's mouth when the removable partial denture 2410 is positioned in the patient's mouth and when the gingival shroud 2450 is assembled to the base framework 2420.

The gingival shroud 2450 can be a complementary addition to the original 3D printed framework 2420 or other structure of the removable partial denture 2410 or other intermediary structure. The complementary mating structure of the gingival shroud 2450 design allows the gingival shroud 2450 to fit over and around the tooth segments 2430 that have already been put into place either as part of the original RPD framework 2420 or added later in process. The RPD framework 2420 can include holes 2440 that receive complementary mating protruding cylinders 2465 on the gingival shroud 2450 for attaching the gingival shroud 2450 to the RPD framework 2420. Alternatively, the gingival shroud can include holes that receive complementary mating protruding cylinders of the RPD framework for attaching the gingival shroud to the RPD framework. These holes and complementary mating protruding cylinders can allow for some mechanical undercut which would allow for a snap sensation upon placement and also provide for a mechanical interlock with the bonding or luting agents utilized in the process for forming the removable partial denture 2410. These holes and complementary mating protruding cylinders can allow for proper orientation of the gingival shroud 2450 and can be locked in place with a slight undercut area in order to achieve a simple snap to place sensation. A chemical bonding agent, light activated bonding agent, or mechanical luting system or chemical luting system can be used to lock the gingival shroud 2450 and the RPD framework 2420 or other intermediary or finished RPD together once these holes and complementary mating protruding cylinders of the gingival shroud and the RPD framework (or intermediary structure) have been oriented properly. FIG. 24A shows in top view in a patient's mouth, the gingival shroud 2450 assembled to the 3D printed removable partial denture 2410.

### Integral Tooth Segment and Clasp Retainer

A method of the invention that makes for easy 3D printing of digital RPDs is to make a white tooth segment in a pink base, but also include the clasp retainer as one piece with the tooth segment. Turning to FIG. 25, there is shown a removable partial denture 2510 having a base 2520 with tooth segments 2530. In the removable partial denture 2510, the white tooth segments 2530 can be fabricated as one piece with a clasp retainer 2540. In this method, the clasp retainer 2540 and the tooth segment 2530 are one piece (possibly made from flexible resin or acetal) and slide into or through the pink base 2520. An opening 2570 is left for the clasp retainer 2540 to slide through or go over the base 2520 as shown in FIG. 25. One advantage of this method is that two simple pieces can be easily 3D printed and assembled while combining the largest advantages of modern materials and in office small scale SLA printing.

In the tooth horn method of FIG. 25 (single piece tooth segment/clasp retainer), the clasp retainer/tooth segment may fit into a corresponding opening in the base and the clasp retainer portion may fit through a hole 2580 in the base 2520 to add to the gingival aesthetic in the gingival embrasure area of the abutment tooth. The clasp retainer part of the tooth segment/clasp retainer piece could mate in the opening in the base, sit on top of the base, or pass through the hole 2580 in the base.

### Example

The following example is provided in order to demonstrate and further illustrate certain embodiments and aspects of the present disclosure and is not to be construed as limiting the scope of the disclosure.

Our experimentation with digital removable partial dentures began with our attempts to make RPD (removable partial denture) frameworks on site rather than outsourcing them to commercial laboratories. This is essentially the center of the movement in dentistry toward 3D printing in general. Dentists and clinics enjoy the idea of cutting out the middle man and the promise of 3D printing allows in office or on-site production. Largely, this is a matter of cost, convenience, and a general push toward same day dentistry.

Normally, a cobalt chrome RPD framework requires ten working days or two calendar weeks to get back from the dental laboratory. It is very common to need to rush this timeline for scheduling or emergency purposes and this causes additional fees. Thus, we were searching for a solution that produces RPD frames on-site saving time and money.

Our first attempts at this resulted in milling a flexible acetal framework on our 4-axis mill. This procedure requires relatively simple, cost effective equipment to produce the frame, but also required the traditional method of setting teeth in wax on an articulator and conventional heat cured acrylic processing (warping and shrinkage along with stone models). Although milling the flexible acetal frame on site saved time, it did not completely streamline and digitize the process.

Our next attempts were aimed at further digitizing the process. The idea that is paramount in digital dentistry is the ability to scan and design the prosthetics from intraoral scan data only. This eliminates completely the need for stone models. In order to achieve this lofty goal with the removable partial denture, the next logical step was to somehow eliminate the need for setting the teeth in wax and conventional acrylic processing eliminating the stone model required to process acrylic to. Therefore, we started to incorporate the tooth colored acetal teeth into the acetal frame design resulting in a one-piece RPD design. This is where many other people developing methods are still at, with a single piece acrylic or acetal design.

While this single piece method worked well for smaller edentulous spaces and completely tooth borne appliances, it had structural and aesthetic limitations. The aesthetic limitations could be corrected in further later attempts with the layering of composite stains on the acetal teeth. However, it was found to discolor easily in some cases from wine, coffee, and smoking, and delaminate at varying rates depending on the application method. Mostly these limitations led to limited marketability and patient satisfaction. For that reason, it was not often prescribed, although it could be done completely from intraoral scanning.

At this point, it was believed that there was a market demand for a digital RPD solution because the acetal frames were working very well and catching on quickly. However, it was also obvious that the acetal frame method still required a stone model and traditional steps. The single piece acetal method improved on the clinical and laboratory workflows, but it had its own problems with aesthetics and rigidity of the major connector for large edentulous spaces. Therefore, our focus changed to yet a third solution.

The next solution and the current proposed method produces a two part RPD with a frame (metal, acetal, PEEK, etc.) and an (acrylic) tooth segment. The design modifications that are unique are made to each printed or milled piece and include a third Boolean subtraction design for a series of resin channels as a retention element that is cut out as negative space from both pieces. The resulting prosthetic meets all the criteria for the ideal digital removable prosthetic. It can be made from an intraoral scan alone. It can be made in a small number of appointments. It requires no shipping by the dentist at all. It can be 3D printed (e.g., by fused deposition modeling, stereolithography, or selective laser sintering) or milled. It can be made from several materials. It can include a rigid or flexible major connector from the same design. It requires no stone model for assembly. Most importantly, the new design results in the most marketable RPD that can be priced to match current models for the dentist.

Although the invention has been described in considerable detail with reference to certain embodiments, one skilled in the art will appreciate that the present invention can be practiced by other than the described embodiments, which have been presented for purposes of illustration and not of limitation. Therefore, the scope of the appended claims should not be limited to the description of the embodiments contained herein.

1. A removable partial denture comprising:
a frame sized and shaped to conform to a mouth inner surface, the frame including a projecting mounting structure; and
a segment having a slot for slidably receiving the projecting mounting structure when the segment is assembled to the frame.

2. (canceled)

3. (canceled)

4. The removable partial denture of claim 1 wherein:
the projecting mounting structure comprises an I-beam.

5. The removable partial denture of claim 4 wherein:
a longitudinal axis of the I-beam extends distally to mesially.

6. The removable partial denture of claim 4 wherein:
a longitudinal axis of the I-beam extends buccally to lingually.

7. The removable partial denture of claim 4 wherein:
the I-beam comprises opposed end plates connected by a midsection,
the midsection includes a channel,
the tooth segment includes a throughhole, and
the channel and the throughhole are aligned when the tooth segment is assembled to the frame.

8. The removable partial denture of claim 7 further comprising:
a fastener positioned in the channel and the throughhole for immobilizing the tooth segment on the frame when the tooth segment is assembled to the frame.

9. The removable partial denture of claim 8 wherein:
the fastener comprises a cured resin.

10. The removable partial denture of claim 8 wherein:
the fastener comprises a pin.

11. (canceled)

12. (canceled)

13. (canceled)

14. The removable partial denture of claim 1 wherein:
the frame includes at least one additional projecting mounting structure, and
the removable partial denture includes at least one additional segment, each additional segment having a slot for slidably receiving one of the additional projecting mounting structures when the additional segments are assembled to the frame.

15. (canceled)

16. The removable partial denture of claim 1 further comprising:
a clasp retainer attached to the frame.

17. The removable partial denture of claim 16 wherein:
the clasp retainer comprises an I bar clasp.

18. The removable partial denture of claim 16 wherein:
the clasp retainer comprises a circumferential clasp.

19. The removable partial denture of claim 16 wherein:
the clasp retainer comprises a wrought wire clasp.

20. The removable partial denture of claim 19 wherein:
the wrought wire clasp is embedded in a base section of the frame, the base section being below a tooth segment.

21. The removable partial denture of claim 20 wherein:
the wrought wire clasp includes a section that matingly engages corresponding structure in the base section of the frame.

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. A removable partial denture comprising:
a frame sized and shaped to conform to a mouth inner surface;
a tooth segment assembled to the frame; and
a wrought wire clasp including a section that matingly engages corresponding structure in the frame or the tooth segment.

40. The removable partial denture of claim 39 wherein:
the section of the wrought wire clasp matingly engages corresponding structure in the frame.

41. The removable partial denture of claim 39 wherein:
the section of the wrought wire clasp matingly engages corresponding structure in the tooth segment.

42. (canceled)

43. (canceled)

44. (canceled)

45. (canceled)

46. A removable partial denture comprising:
a base sized and shaped to conform to an inner surface of a patient's mouth;
a tooth segment attached to the base; and
a removable gingival shroud including a section that matingly engages corresponding structure in the base when the gingival shroud is assembled to the base or a frame of the removable partial denture.

47. The removable partial denture of claim 46 wherein:
the section of the gingival shroud includes one or more protrusions, and
the corresponding structure of the base defines one or more holes for receiving the one or more protrusions.

48. (canceled)

49. (canceled)

50. (canceled)

51. (canceled)

52. (canceled)

53. (canceled)

54. (canceled)

55. (canceled)

56. (canceled)

57. A removable partial denture comprising:
a base sized and shaped to conform to an inner surface of a patient's mouth;
a tooth segment attached to the base; and
a clasp retainer,
wherein the tooth segment and the clasp retainer are formed as one piece.

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)
